(19) United States
(12) Patent Application Publication
(10) Pub. No.: US 2010/0050322 A1
(43) Pub. Date: Mar. 4, 2010

(54) Orthotic footsock and integrated removable gel arch pad

(76) Inventor: Matthew James Zagula, Georgetown, KY (US)

Correspondence Address: STOLL KEENON OGDEN PLLC, 300 WEST VINE STREET, SUITE 2100, LEXINGTON, KY, 40507-1801, US

(21) Appl. No.: 12/552,266

(22) Filed: Sep. 1, 2009

Related U.S. Application Data
(60) Provisional application No. 61/093,717, filed on Sep. 2, 2008.

(51) Int. Cl. A43B 17/00; A41D 27/20; A61F 5/14

(52) U.S. Cl. 2/239; 2/247; 36/153

(57) ABSTRACT

An orthotic device having an arch gel pad formed of resilient material. The gel pad is designed to be removably placed into and housed within an elongated pocket which sewn into the interior of a foot sock and along the sock bottom, wherein the arch gel pad pocket is capable of housing the arch gel pad.

### RELATED APPLICATIONS

The present application claims priority from parent application U.S. Provisional Pat. App. Ser. No. 61/093,717 and relates to and claims priority with regard to all common subject matter of the underlying utility patent application filed Sep. 2, 2009. The identified provisional patent application is hereby wholly incorporated into the present application by reference.

### BACKGROUND OF THE INVENTION

1. Field of the Invention

This invention relates to orthopedic devices. More specifically this invention relates to orthopedic inserts. Even more specifically, this invention relates to socks with orthopedic inserts.

2. Problems in the Art

The foot is a combination of 26 bones, 33 joints, 112 ligaments, and a network of tendons, nerves, and blood vessels that all combine to support the human frame during climbing, standing, running, and walking. Orthotics are shoe inserts that are intended to correct an abnormal, or irregular, walking or running pattern. Orthotics are not arch supports. They perform functions that make standing, walking, and running more comfortable and efficient by altering the angles at which the foot strikes a walking or running surface. This allows feet and lower legs to function at their highest potential by properly aligning the muscles, tendons and bones of the foot. Orthotics can improve the user's quality of life by decreasing pain and preventing injury by increasing stability.

Foot orthotics fall into three broad categories: those that primarily attempt to change foot function, those that are primarily protective in nature, and those that combine functional control and protection. Rigid orthotic devices are designed to control foot function, and may be made from a firm material such as plastic or carbon fiber. These types of orthotics are mainly designed to control motion in two major foot joints, which lie directly below the ankle joint. This type of orthotic is often used to improve or eliminate pain in the legs, thighs and lower back due to abnormal function of the foot. Ideally, such an orthotic is custom fitted and sometimes created from molds of the wearer's feet.

Soft orthotic devices help to protect the foot and work to reduce pressure and minimize shock. Soft orthotics are usually made of soft, compressible materials. This type of orthotic is effective for arthritis or deformities where there is a loss of protective fatty tissue on the side of the foot. They are also helpful for people with diabetes.

Semi-rigid orthotics are used to provide control and as well as protection. They are often used by athletes or those who spend a considerable amount of time on their feet. It allows for responsive support of the foot while standing, running or participating in sports. By guiding the foot through proper functions, it allows the muscles and tendons to perform more efficiently. It is constructed of layers of soft materials, reinforced with more rigid materials.

Podiatrists, physical therapists and sports medicine practitioners will often recommend custom foot orthoses as part of a treatment regimen. They are prescribed to reduce the symptoms associated with many foot related pathologies. Custom orthotics can be expensive.

A prescription foot orthosis is an in-shoe brace which is designed to correct for abnormal foot and lower extremity function [the lower extremity includes the foot, ankle, leg, knee, thigh and hip]. In correcting abnormal foot and lower extremity function, the prescription foot orthosis reduces the strain on injured structures in the foot and lower extremity, allowing them to heal and become non-painful. In addition, prescription foot orthoses help prevent future problems from occurring in the foot and lower extremity by reducing abnormal or pathological forces acting on the foot and lower extremity. A prescription foot orthoses is more commonly known by the public as a “foot orthotic”.

Podiatrists prescribe two main types of prescription foot orthoses for their patients, accommodative orthoses and functional foot orthoses. Both types of prescription foot orthoses are used to correct the foot plant of the patient so that the pain in their foot or lower extremity will improve so that normal activities can be resumed without pain. However, accommodative and functional foot orthoses are generally made using different materials and may not look or feel the same. Both types of prescription foot orthoses are nearly always prescribed as a pair to allow more normal function of both feet [similar to having both the left and right wheels of a car realigned in a front end alignment]. Accommodative foot orthoses are used to cushion, pad or relieve pressure from a painful or injured area on the bottom of the foot. They may also be designed to try to control abnormal function of the foot. Accommodative orthoses may be made of a wide range of materials such as cork, leather, plastic foams, and rubber materials. They are generally more flexible and soft than functional foot orthoses. Accommodative orthoses are fabricated from a three dimensional model of the foot which may be made by taking a plaster mold of the foot, stepping into a box of compressible foam, or scanning the foot with a mechanical or optical scanner.

Accommodative orthoses are useful in the treatment of painful callouses on the bottom of the foot, diabetic foot ulcerations, sore bones on the bottom of the foot and other types of foot pathology. The advantages of accommodative orthoses are that they are relatively soft and forgiving and are generally easy to adjust in shape after they are dispensed to the patient to improve comfort. The disadvantages of accommodative orthoses are that they are relatively bulky, have relatively poor durability, and often need frequent adjustments to allow them to continue working properly.

The advantages of prescription foot orthoses are many. First of all, they are custom made for each foot of each patient, so that each foot orthosis will only fit one foot correctly. In addition, since they fit so exactly to the persons foot, they can be made with relatively rigid, durable materials with a minimal chance of discomfort or irritation to the patients foot. Prescription foot orthoses also have a much greater potential to effectively and permanently treat painful conditions, all the way from the toes to the lower back, since they are designed specifically for an individual's biomechanical nature.

Functional foot orthoses are used to correct abnormal foot function and, in so doing, also correct for abnormal lower extremity function. Some types of functional foot orthoses may also be designed to accommodate painful areas on the bottoms of the foot, just like accommodative foot orthoses. Functional foot orthoses may be made of flexible, semi-rigid or rigid plastic or graphite materials. They are relatively thin and easily fit into most types of shoes. They are fabricated from a three dimensional model of the foot which may be made by taking a plaster mold of the foot, stepping into a box of compressible foam, or scanning the foot with a mechanical or optical scanner.

Functional foot orthoses are useful in the treatment of a very wide range of painful conditions of the foot and lower extremities. Big toe joint and lesser toe joint pain, arch and instep pain, ankle pain and heel pain are commonly treated with functional foot orthoses. Since abnormal foot function causes abnormal leg, knee and hip function, then functional foot orthoses are commonly also used to treat painful tendinitis and bursitis conditions in the ankle, knee and hip, in addition to shin splints in the legs. The advantages of functional foot orthoses are that they are relatively durable, infrequently require adjustments and more likely to fit into standard shoes. The disadvantages are that they are relatively difficult to adjust and relatively firm and less cushiony.

For example, in children, prescription foot orthoses are used to prevent abnormal development of the foot due to flatfoot or intoeing or outtoeing disorders. In athletes, prescription foot orthoses are used to allow the athlete to continue training and competing without pain. And in most adult patients, prescription foot orthoses are used to allow more normal daily activities without pain or disability.

One disadvantage to prescription foot orthoses is that they are relatively expensive when compared to store bought over-the-counter foot inserts. Even though the over-the-counter inserts do help some people with mild symptoms, they do not have the potential to correct the wide range of symptoms that prescription foot orthoses can since they are made to fit a person with an “average” foot shape.

An additional foot problem is called runner's toe. Bleeding under the toenail usually occurs from the toenail repeatedly making contact with the shoe. This causes bleeding under the toenail from a shearing force that can separate the toenail from the nail bed (skin holding the nail to the toe). This often happens because shoes are too tight or because toenails are too long. It can also happen if a heavy object strikes the toenail.

Currently no orthotic device is known to the inventor that incorporates a foot sock with an insertable orthotic support placed within a pocket with the sock. Also, no known orthotic device also attempts to correct runner's toe by the insertion of a toe gel pad within the sock or integrated into the toe of the sock.

### BRIEF DESCRIPTION OF THE DRAWINGS

FIG. 1 provides a perspective view of an arch gel pad

FIGS. 2a and 2b depict a two cross sectional views of the gel pad, revealing the flat, bottom gel layer and top gel layer.

FIG. 3 depicts a cut-away view of a sock revealing the interior pocket along bottom of the sock at the arch.

FIG. 4 is a side profile view of the arch gel pad in an arch gel pad pocket engaging and conforming to the arch of the wearer's foot.

### DESCRIPTION OF THE INVENTION

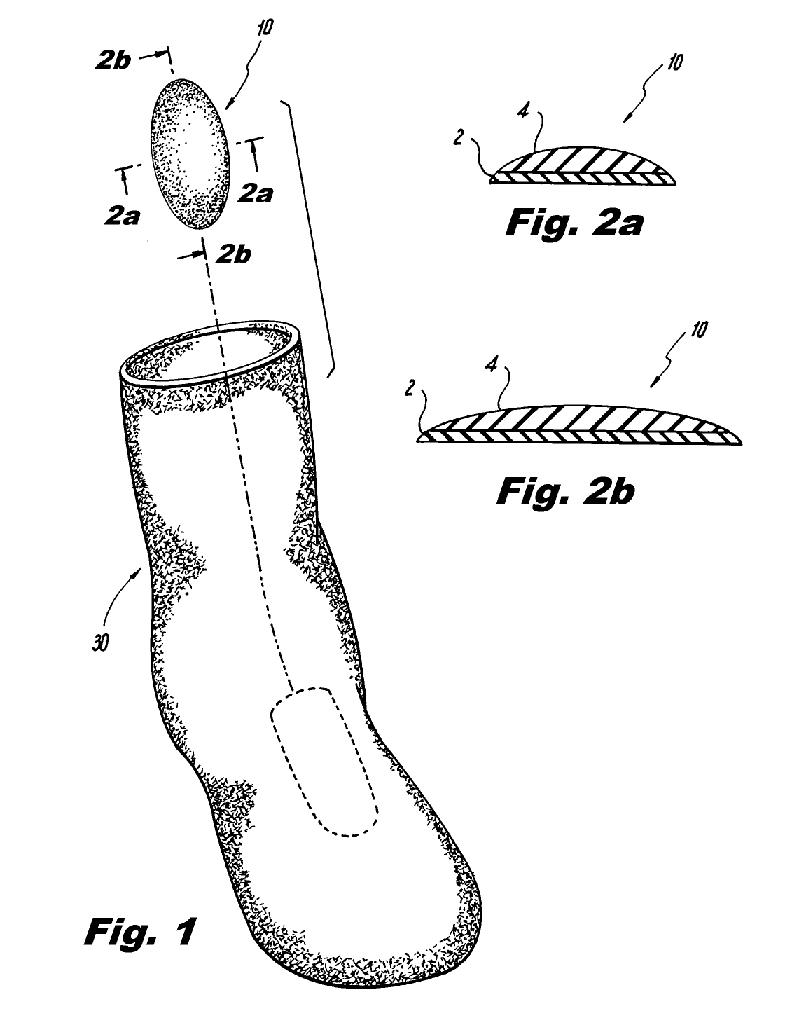

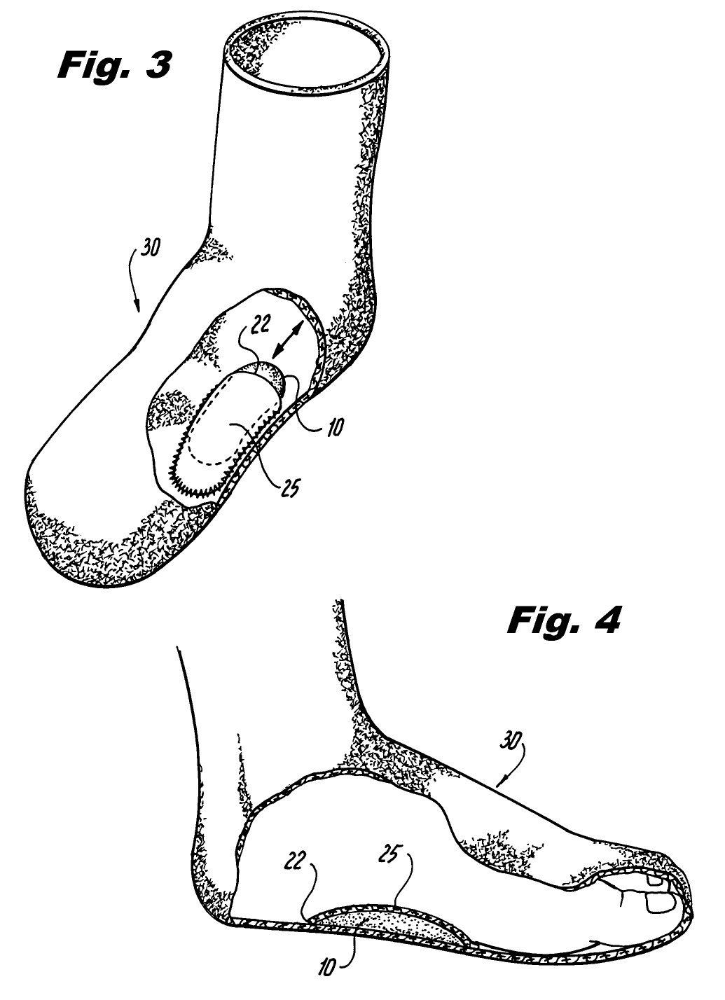

The present invention incorporates an orthotic silicone gel pad 10, as depicted in FIG. 1, within a sock 30. As seen in FIG. 3, the sock preferably possesses an elongated gel pad pocket 25 sewn into the inside of the sock 30 into which a gel pad is inserted through a pocket mouth 22. Preferably the arch gel pad pocket 25 is approximately 33% larger than the volume of the arch gel pad 10. The gel pad pocket could be larger or smaller. The pocket preferably runs lengthwise along the arch area 28 of the sock 30 and is substantially oblong in shape with the height of the arch gel pad pocket 25 being greater than the width of the pocket, so as to inhibit the arch gel pad 10 from rolling.

The arch gel pad 10 is preferably comprised of at least two layers, a top gel layer 4 and a bottom gel layer 2. The top gel layer 4, near the foot, is comprised of a softer gel having a relatively low density so as to allow the arch gel pad 10 to conform comfortably to the arch of the foot when the sock 30 is worn, as is shown in FIG. 4. The purpose of the top gel layer 4 is to absorb some of the shock normally transferred between the shoe and the foot. The lower gel layer 2 is preferably formed from a higher density silicone than the top gel layer 4 so as to provide structural support to the arch gel pad 10. The arch gel pad 10 possesses a greater width than height so as to inhibit rolling of the arch gel pad 10 within the gel pad pocket 25.

As shown in FIGS. 2a and 2b, the arch gel pad 10 is substantially flat across its base, formed of the bottom gel layer 2, and preferably possesses a length greater than its width. The top gel layer 4 is rises from the bottom gel layer 2 and is curved so that its height rises in a curvilinear manner from the bottom gel layer 2 to the point at which it crests, typically at a point roughly at the mid-point of the gel arch pad 20 when viewed from above. The shape of the arch gel pad 10 is designed to fit within the arch of the human foot and is curved to aid in comfort.

The arch gel pad 10 is formed from resilient materials. The arch gel pad 10 is preferably formed by introducing a higher density resin and a lower density resin into a mold. The layered resins in the mold are then cured. Curing can be accomplished by the appropriate method for the resin selected. Silicone is preferred as a resilient material because it is a poor medium for the growth of bacteria and fungus. Optionally, bactericides and/or fungicides could either be integrated into the resilient material of the arch gel pad 10 or coated onto the surface of the arch gel pad 10. Bactericides and/or fungicides could potentially comprise nanoparticles such as nano-silver particles. Metallic nanoparticles, such as silver, are preferred due to known bactericidal properties.

The arch gel pad 10 is layered so that the soft, top gel layer 4 is affixed to the top of the firmer bottom gel layer 2. This provides a soft, yet firm support near the arch while the gel foundation anchors the top gel layer 4 to inhibit drastic deformation. The top gel layer 4 acts not only to soften the feel, but to dampen vibration and to distribute pressure evenly along the arch of the wearer's foot. The top gel layer 4 is thickest at the substantially the center point of the arch gel pad 10 width and length, which permits the arch gel pad 10 to deform along its width and length to accommodate the shape of the wearer's arch. The arch gel pad 10 is can be coated with talc to facilitate insertion into and removal from the arch gel pad pocket 25.

Preferably, additional cross-stitching is used to reinforce the arch. Structural support is provided by reducing the flexibility of the sock along the arch and restricting the stretch of the sock around the circumference of the foot along the arch.

What is claimed is:

1. A device comprising:
(a) an arch gel pad formed of resilient material;
(b) an arch gel pad pocket within the interior of a foot sock and along the sock bottom, wherein said arch gel pad pocket is capable of housing said arch gel pad.

2. The device of claim 1, wherein said arch gel pad pocket is configured to allow the removal and insertion of said arch gel pad into said arch gel pad pocket.

3. The device of claim 1, wherein said arch gel pad pocket runs along the length of the arch of said foot sock.

4. The device of claim 3, wherein said arch gel pad pocket has a width greater than its height.

5. The device of claim 1, wherein said arch gel pad has length, width, height, a substantially flat base, an arched top, and having a shape, formed by its perimeter, which substantially forms an oblong circle along its length when contrasted against its width.

6. The device of claim 5, wherein said top of said arch gel pad support conforms to and engages the arch of the human foot.

7. The device of claim 6, wherein said height of said arch gel pad is greatest near the center of said arch gel pad and gradually decreases towards the perimeter.

8. The device of claim 7, wherein said base of said arch gel pad is substantially flat.

9. The foot sock of claim 1, wherein said gel arch gel pad is comprised of polymerized silicone.

10. The foot sock of claim 9, wherein said arch gel pad is comprised of a substantially flat bottom gel layer of polymerized silicone and a substantially arched top gel layer of silicone which rises from said substantially flat bottom gel layer.

11. The device of claim 10, wherein said bottom gel layer of silicone has a greater shore density that said top gel layer of silicone.

12. The device of claim 1, wherein said arch gel pad pocket is approximately 33% longer than said arch gel pad.

13. The method of creating an orthotic device by inserting a gel orthotic support into a pocket within a foot sock.

14. An arch gel pad formed of resilient materials, wherein said arch gel pad is intended for insertion into a gel pad pocket of a foot sock.

15. The device of claim 14, wherein said arch gel pad has length, width, height, a substantially flat base, an arched top, and a perimeter which substantially forms an oblong circle along its length when contrasted against its width.

16. The device of claim 15, wherein said height of said arch gel pad is greatest near the center of said arch gel pad and gradually decreases towards the perimeter.

17. The device of claim 16, wherein said top of said arch gel pad support conforms to and engages the arch of the human foot.

18. The device of claim 17, wherein said base of said arch gel pad is substantially flat.

19. The foot sock of claim 18, wherein said arch gel pad is comprised of polymerized silicone.

20. The foot sock of claim 19, wherein said arch gel pad is comprised of a substantially flat bottom gel layer of polymerized silicone and a substantially arched top gel layer of silicone which rises from said substantially flat bottom gel layer.
